(19) United States
(12) Patent Application Publication
(10) Pub. No.: US 2005/0089936 A1
(43) Pub. Date: Apr. 28, 2005

(54) Combinatorial library of 3-aryl-1H-indole-2-carboxylic acid amides

(76) Inventors: Jianping Cai, West Caldwell, NJ (US); Robert Alan Goodnow JR., Gillette, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 10/957,161

(22) Filed: Oct. 1, 2004

Related U.S. Application Data
(60) Provisional application No. 60/513,785, filed on Oct. 23, 2003.

(51) Int. Cl. G01N 33/53; C07D 471/02; A61K 31/4745; A61K 31/405

(52) U.S. Cl. 435/7.1; 514/300; 514/419; 546/113; 548/492

(57) ABSTRACT

Combinatorial libraries that contain various different 4,5-fused-3-substituted-2-pyrrocarboxylic amides for screening pharmacological activity and methods of synthesizing said libraries.

### PRIORITY TO RELATED APPLICATIONS

This application claims the benefit of Provisional Application Ser. No. 60/513,785, filed Oct. 23, 2003.

### TECHNICAL FIELD

This invention is directed to combinatorial chemistry libraries containing 3-aryl-2-indolylcarboxamides as well as solid phase methods for constructing such combinatorial chemistry libraries.

### BACKGROUND

Modern day drug discovery is a multi-faceted endeavor. Researchers commonly delineate a biochemical pathway that is operative in a targeted pathological process. This pathway is analyzed with an eye toward determining its crucial elements: those enzymes or receptors that, if modulated, could inhibit the pathological process. An assay is constructed such that the ability of the important enzyme or receptor to function can be measured. The assay is then performed in the presence of a variety of molecules. If one of the assayed molecules modulates the enzyme or receptor in a desirable fashion, this molecule may be used directly in a pharmaceutical preparation or can be chemically modified in an attempt to modulate its beneficial activity. The identified molecule that exhibits the best profile of beneficial activity may ultimately be formulated as a drug for the treatment of the targeted pathological process.

With the use of high-throughput screening techniques, one can assay the activity of tens of thousands of molecules per week. Where molecules can only be synthesized one at a time, the rate of molecule submission to an assay becomes a debilitating, limiting factor. This problem has led researchers to develop methods by which large numbers of molecules possessing diverse chemical structures can be rapidly and efficiently synthesized. One such method is the construction of chemical combinatorial libraries.

Chemical combinatorial libraries are diverse collections of molecular compounds. Gordon et al. (1995) Acc. Chem. Res. 29:144-154. These compounds are formed using a multi-step synthetic route, wherein a series of different chemical modules can be inserted at any particular step in the route. By performing the synthetic route multiple times in parallel, each possible permutation of the chemical modules can be constructed. The result is the rapid synthesis of hundreds, thousands, or even millions of different structures within a chemical class.

For several reasons, the initial work in combinatorial library construction focused on peptide synthesis. Furka et al. (1991) Int. J. Peptide Protein Res. 37:487-493; Houghton et al. (1985) Proc. Natl. Acad. Sci. USA 82:5131-5135; Geysen et al. (1984) Proc. Natl. Acad. Sci. USA 81:3998; and Fodor et al. (1991) Science 25:767. The rapid synthesis of discrete chemical entities is enhanced where the need to purify synthetic intermediates is minimized or eliminated; synthesis on a solid support serves this function. Construction of peptides on a solid support is well known and well documented. Obtaining a large number of structurally diverse molecules through combinatorial synthesis is furthered where many different chemical molecules are readily available. Finally, many peptides are biologically active, making them interesting as a class to the pharmaceutical industry.

The scope of combinatorial chemistry libraries has recently been expanded beyond peptide synthesis. Polycarbamate and N-substituted glycine libraries have been synthesized in an attempt to produce libraries containing chemical entities that are similar to peptides in structure, but possess enhanced proteolytic stability, absorption and pharmacokinetic properties. Cho et al. (1993) Science 261:1303-1305; Simon et al. (1992) Proc. Natl. Acad. Sci. USA 89:9367-9371. Furthermore, benzodiazepine, pyrrolidine, and diketopiperazine libraries have been synthesized, expanding combinatorial chemistry to include heterocyclic entities Bunin et al. (1992) J. Am. Chem. Soc. 114:10997-10998; Murphy et al. (1995) J. Am. Chem. Soc. 117:7029-7030; and Gordon et al. (1995) Biorg. Medicinal Chem. Lett. 5:47-50.

Substituted indoles are a class of bioactive, heterocyclic molecules that have attracted considerable attention in the pharmaceutical industry. Bunker, Edmunds et al., Bioorg. Med. Chem. Lett. 1996, 6(9), 1061-66.

Methods for the solution phase preparation of 3-aryl-2-indolylcarboxic acid amides have been reported. Ger. Offenlegungschrift 1,812,205, Sumitomo Chemical Co. ltd.; Chem. Abstr., 71, 124521 F (1969); Zeeh, B. Chem. Ber. 1969, 102, 678-685. In this method, two equivalents of an isonitrile are condensed with one equivalent of diarylketone with boron trifluoride catalyst. This route is limited to the production of particularly substituted 3-aryl-2-indolylcarboxamides. The solution phase chemistry method also has practical limitations, which hinders the synthesis of thousands of analogs that are possible with a solid phase synthesis approach.

### SUMMARY OF THE INVENTION

The present invention is directed to a combinatorial library containing a plurality of different compounds of various structures within the formula: 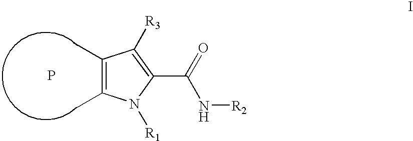
wherein P is a fused ring substituent, which ring substituent is an aromatic ring, a heteroaromatic ring or a cycloaliphatic ring which may be substituted or unsubstituted; R₁ and R₂ are individually hydrogen, lower alkyl containing from 1 to 7 carbon atoms, lower alkenyl containing from 2 to 7 carbon atoms, lower alkynyl containing from 3 to 7 carbon atoms, mono or bicycloaliphatic ring with each ring having from 3 to 7 carbon atoms, aryl containing from 1 to 3 fused aromatic rings each ring consisting of 6 carbon atoms, heterocyloaliphatic containing 1 to 2 fused rings with each ring containing from 3 to 6 carbon atoms with one or two hetero atoms selected from the group consisting of O, S and N, monocyclic or bicyclic heteroaryl rings each containing from 3 to 6 carbon atoms with 1 to 4 hetero atoms which can be N, S or O with the proviso that when the hetero atom is S or O, there are 1 or 2 hetero atoms in the ring and when the hetero atoms is N, there are from 1 to 4 N atoms in the ring, and wherein the hetero ring in the heterocycloaliphatic ring or monocyclic or bicyclic heteroaryl rings can be condensed with an aryl, or cycloaliphatic ring and wherein any of the heteroaryl, aryl cycloaliphatic or heteroaliphatic rings in the cyloaliphatic aryl, heteroaryl or heteroaliphatic substituents may be connected to the formula I by a lower alkylene chain containing from 1 to 7 carbon atoms and R₃ is a ring substituent selected from the group consisting of an aromatic ring, a heteroaromatic ring and a cycloaliphatic ring which ring substituent may be substituted or unsubstituted.

The library of compounds is prepared by first reacting a compound of the formula: 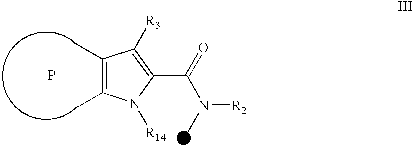
wherein ● is a solid support, R₁₃ is a leaving group; R₁₄ is an amino protecting group or R₁₇, and R₁₇ is R₁ other than hydrogen and, R₂, R₁ and P are as above,
with a boronic acid of the formula: 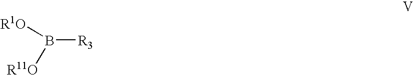
wherein R₃ is as above R¹ and R¹¹ are individually lower alkyl or taken together form a lower alkylene bridge between their attached oxygen atoms,
   to produce an immobilized compound of the formula: 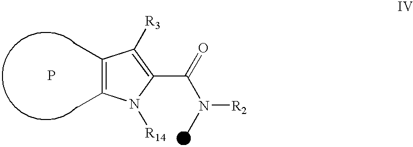
wherein ●, P, R₁₄, R₂, and R₃ are as above.

The compound of formula IV can be cleaved by the methods mentioned hereinafter, such as hydrolysis or photolytic cleavage, from the solid support to produce the compound of formula I. Where R₁₄ is an amino protecting group, hydrolysis will produce the compound of formula I where R₁ is hydrogen. When R₁₄ is R₇, which is R₁, other than hydrogen, it will produce the compound of formula I where R₁ is other than hydrogen.

It has been discovered, in accordance with this invention, that the combinatorial library of different compounds having the formula I is produced simply and readily in high yields by ensuring that the N atom in the 1-position of the indole ring be either protected or derivatized with a substituent designated by R₁₄. The protection or derivatization will allow the substituent R₃ to be placed at the 3-position on the indole ring through the reaction with the boronic acid compound of formula V. In this manner, a series of different compounds of the formula I can be easily produced with various boronic acids of formula V to build up a combinatorial library.

### DETAILED DESCRIPTION

The present invention provides a combinatorial library that contains various different 4,5-fused-3-substituted-2-pyrrolocarboxamides, where the P ring is an aromatic ring, a heteroaromatic ring, an aliphatic ring or substituted versions thereto, of the formula: 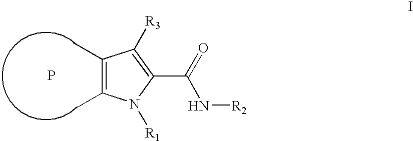
wherein P, R, R₂ and R₃ are as above.

In one embodiment, the chemical method for the production of combinatorial library compounds contains methodology for the solid phase synthesis of 3-substituted-2-indolyl-carboxamides. The compounds which make up the library include but are not limited to the following.
R₁-3-R₃-1H-indole-2-carboxylic acid R₂ amide
7-R₁-5-R₃-7H-pyrrolo[2,3-d]pyrimidine-6-carboxylic acid R₂ amide
5-R₁-7-R₃-5H-pyrrolo[3,2-d]pyrimidine-6-carboxylic acid R₂ amide
R₁-3-R₃-1H-pyrrolo[3,2-c]pyridine-2-carboxylic acid R₂ amide
R₁-3-R₃-1H-pyrrolo[2,3-c]pyridine-2-carboxylic acid R₂ amide
R₁-3-R₃-1H-pyrrolo[2,3-b]pyridine-2-carboxylic acid R₂ amide
R₁-3-R₃-1H-pyrrolo[3,2-b]pyridine-2-carboxylic acid R₂ amide
5-R₁-7-R₃-5H-pyrrolo[2,3-b]pyrazine-6-carboxylic acid R₂ amide
R₁-3-R₃-1H-pyrrolo[2,3-d]pyridazine-2-carboxylic acid R₂ amide
6-R₁-4-R₃-6H-thieno[2,3-b]pyrrole-5-carboxylic acid R₂ amide
6-R₁-4-R₃-6H-furo[2,3-b]pyrrole-5-carboxylic acid R₂ amide
4-R₁-6-R₃-4H-furo[3,2-b]pyrrole-5-carboxylic acid R₂ amide
4-R₁-6-R₃-4H-thieno[3,2-b]pyrrole-5-carboxylic acid R₂ amide.

In accordance with various embodiments of this invention, for 1-R₁-3-R₃-1H-indole-2-carboxylic acid R₂ amide, 7-R₁-5-R₃-7H-pyrrolo[2,3-d]pyrimidine-6-carboxylic acid R₂ amide, 5-R₁-7-R₃-5H-pyrrolo[3,2-d]pyrimidine-6-carboxylic acid R₂ amide, 1-R₁-3-R₃-1H-pyrrolo[3,2-c]pyridine-2-carboxylic acid R₂ amide, 1-R₁-3-R₃-1H-pyrrolo[2,3-c]pyridine-2-carboxylic acid R₂ amide, 1-R₁-3-R₃-1H-pyrrolo[2,3-b]pyridine-2-carboxylic acid R₂ amide, 1-R₁-3-R₃-1H-pyrrolo[3,2-b]pyridine-2-carboxylic acid R₂ amide, 5-R₁-7-R₃-5H-pyrrolo[2,3-b]pyrazine-6-carboxylic acid R₂ amide, 1-R₁-3-R₃-1H-pyrrolo[2,3-d]pyridazine-2-carboxylic acid R₂ amide 6-R₁-4-R₃-6H-thieno[2,3-b]pyrrole-5-carboxylic acid R₂ amide, 6-R₁-4-R₃-6H-furo[2,3-b]pyrrole-5-carboxylic acid R₂ amide, 4-R₁-6-R₃-4H-furo[3,2-b]pyrrole-5-carboxylic acid R₂ amide, 4-R₁-6-R₃-4H-thieno[3,2-b]pyrrole-5-carboxylic acid R₂ amide (FIG. 1) where R₄, R₅, R₆, and R₇ can independently be alkyl, aryl, heteroaryl, and electron withdrawing groups. Preferably, the combinatorial library contains a 4,5-fused-3-substituted-2-pyrrolocarboxamides including but not limited to 1-R₁-3-R₃-1H-indole-2-carboxylic acid R₂ amide.

A chemical library is an intentionally created collection of different molecules which can be prepared synthetically and screened for biological activity in a variety of different formats. The library may consist of the soluble molecules themselves or the library can consist of libraries of such molecules bound to a solid support. In both types of formats the combinatorial library of this invention can be screened. The libraries of this invention contain at least two different compounds within the compound of formula I. In general, the libraries of this invention should contain at least 200 different compounds having the structure of Formula I with libraries of from 500 to 10,000 different compounds being preferred. The method of this invention allows one to create a library containing different molecules of the compounds having the formula of formula I. The synthetic chemical route of this invention is ideally suited for mass producing a library of different compounds having the structure of formula I.

Libraries of this invention can be randomized by being deliberately prepared utilizing standard randomization procedures. By these procedures different compounds of formula I, without the R₁ and R₃ substituents can be connected to a solid support and reacted with a cocktail of a mixture of different reagents producing different R₁ and R₃ substituents on the molecule bound to the solid support. The reactions are allowed to proceed so that on each compound on the solid support member is reacted with one of the reactants in this randomized mixture of the reactants. In this manner, a different R₁ and R₃ group will be are placed on each of the various molecules attached to a solid residence support. On the other hand, where the library is deliberately prepared specific reactants which give one specific one R₁ and R₃ substituent are utilized rather than a randomized cocktail of reagents. These specific reagents are specifically geared to producing a given compound on the solid support containing the compound which does not contain any R₁ or R₃ substituent.

As used herein, the term halogen, halo or halide designates all four halogens such as chlorine, bromine, fluorine or iodine. The term lower alkyl designates a saturated monovalent hydrocarbon substituent containing from 1 to 7 carbon atoms such as, for example, methyl, ethyl, n- or iso-propyl or n-, sec-, or tert-butyl or a straight-chain or branched pentyl, hexyl, heptyl substituent. The term lower alkenyl designates an olefinic unsaturated monovalent hydrocarbon substituent containing from 2 to 7 carbon atoms and from 1 to 2 olefinic unsaturated double bonds such vinyl, allyl, 2- or 3-butenyl, isobutenyl or n-penta-2,4-dienyl. The term lower alkynyl designates a monovalent aliphatic acetylenically unsaturated hydrocarbon, containing from 3 to 7 carbon atoms such as 1- or 2-propynyl. The term cycloaliphatic ring designates a monocyclic or bicyclic aliphatic hydrocarbon ring which can be a cyclo lower alkyl or cyclo lower alkenyl ring containing from 3 to 7 carbon atoms. The preferred cyclo lower alkyl ring is a cyclopropyl, dimethylcyclopropyl, cyclobutyl, cyclopentyl or cyclohexyl ring and the preferred cyclo lower alkenyl ring is cyclo pentadienyl or cyclohexenyl ring. The bicyclo alkyl rings consist of two fused alkyl rings, each containing from 3 to 6 carbon atoms such as for example, bornyl or norbornyl.

The term heterocycloaliphatic designates a monovalent cycloaliphatic ring containing from 4 to 5 carbon atoms in the ring with these carbon atoms being interrupted with one or two hetero atoms selected from the group O, S or N. The term aryl designates an aromatic hydrocarbon moiety being from 1, 2 or 3 rings, each ring containing 6 carbon atoms. Where aryl consists of 2 or 3 rings, all of the rings which make up the aryl substituent are fused, which each ring containing 6 carbon atoms. The preferred aryl substituents, other than phenyl, are naphthyl, indenyl, azulenyl or anthryl.

In accordance with this invention, the term heteroaryl designates mono- or bi-cyclic heteroaryl rings each containing from 3 to 6 carbon atoms with 1 to 4 hetero atoms and the hetero atoms in each ring being N, S or O with the proviso that when the hetero atom is S or O, there are 1 or 2 hetero atoms in the ring and when the hetero atoms is N, there are from 1 to 4 nitro atoms in the ring. In accordance with this invention, it is preferred when there are 2 or more hetero atoms in the ring that the hetero atoms be all nitrogen, oxygen or sulfur. Further, in accordance with this invention, the hetero ring in the heterocycloaliphatic or heteroaryl substituent can be fused or condensed with an aryl or cycloaliphatic ring such as defined herein. The preferred aryl is phenyl and the preferred cycloaliphatic rings which are fused with the heteroatom generally should contain only 1 cycloaliphatic ring.

Furthermore, in accordance with this invention, the heteroaryl, cycloaliphatic and heterocyclic ring, when these groups constitute R₁ and R₂ can be connected to their respective N atoms on the compound of formula I by a lower alkylene chain containing from 1 to 7 carbon atoms. The term lower alkylene designated a bivalent saturated hydrocarbon group containing from 1 to 7 carbon atoms. Preferably, the hydrocarbon chain of lower alkylene is a straight-chain which contains a free valence at both the terminal carbon atoms in the chain such as methylene, 1,2-ethylene, 1,3-propylene and 1,4-butylene.

When R₁, R₂, R₃ and P contain aromatic, heteroaromatic or a cycloaliphatic rings, these rings may be substituted or unsubstituted with various substituents, particularity with functional groups or derivatized functional groups. Those functional groups or derivatized functional groups can be amino, C₁-C₄alkylamino, di-C₁-C₄alkylamino, hydroxy, oxo, thio, nitro, carboxy, carbamoyl, sulfo, sulfamoyl, ammonio, amidino, cyano, formylamino, formamido, and halogen or are saturated or unsaturated aliphatic, cycloaliphatic or heterocycloaliphatic radicals, carbocyclic or heterocyclic aryl radicals, or condensed carbocyclic, heterocyclic or carbocyclic-heterocyclic radicals, which may themselves be combined as desired with further such radicals and substituted by the mentioned functional groups or derivatized functional groups. The mentioned substituents and radicals may also be interrupted by one or more bivalent radicals from the group —O—, —S—, —C(═O)O—, —O—C(C═O)—, —C(═O)—N(C₁-C₄alkyl)-, —N(C₁-C₄alkyl)-C(═O)—, —S(═O)—, —S(═O)₂, —S(═O)—O—, S(O)₂—, —S(═O)—N(C₁-C₄alkyl)-, —S(═O)₂—N(C₁-C₄alkyl)-, —(C₁-C₄alkyl)N—S(═O)—, —(C₁-C₄alkyl)N—S(═O)₂—, —P(═O)—, —P(═O)—O—, —O—P(═O)—, and —O—P(═O)—.

In accordance with an embodiment of this invention, the library may contain a plurality of different compounds selected from compounds of the formula: 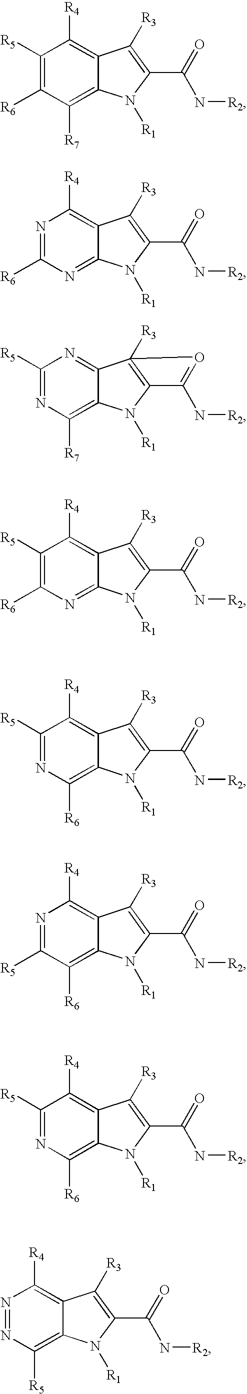 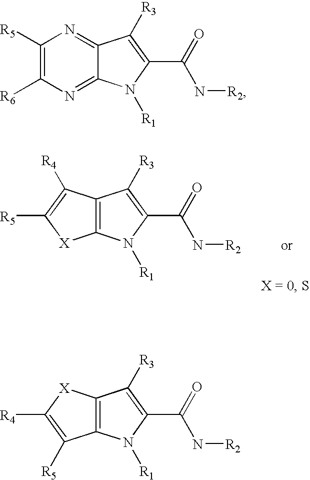
wherein R₁, R₂ and R₃ are as above; and R₄, R₅, R₆ and R₇ are individually selected from functional groups or derivatized functional groups consisting of amino, C₁-C₄alkylamino, di-C₁-C₄alkylamino, hydroxy, oxo, thio, nitro, carboxy, carbamoyl, sulfo, sulfamoyl, ammonio, amidino, cyano, formylamino, formamido, halogen, saturated or unsaturated, cycloalkyl, heterocycloalkyl, aryl, or heteroaromatic rings which may be condensed with aryl, heteroaromatic or heterocycloalkyl rings and X is O or S.

In accordance with another embodiment of this invention, the library may contain a plurality of different compounds where R₃ is selected from the group consisting of 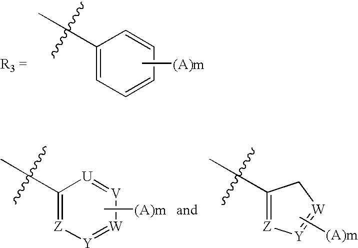
wherein m is an integer of from 1 to 3, A is R₄, R₅, R₆ and R₇ and U, V, W, Y and Z are individually —N—, —O—, —S— or —CH— with at least one of U, V, X or Y being —S—, —O— or —N—.

Suitable substituents A from the group R′₁, R′₂, R′₃, R′₄, and R′₅ are especially functional groups from the group consisting of amino, C₁-C₄alkylamino, for example methyl- or ethyl-amino, di-C₁-C₄alkylamino, for example dimethyl- or diethyl-amino, hydroxy, oxo, thio, nitro, carboxy and halogen, or are substituents from the group lower alkyl, lower alkenyl, lower alkynyl, monocycloalkyl, bicycloalkyl, cycloalkenyl, heterocycloalkyl, aryl, heteroaryl, carbocyclic C₇-C₁₆arylalkyl and heteroarylalkyl, which may themselves be substituted by the mentioned functional groups and interrupted by the mentioned bivalent radicals.

Lower alkyl is, for example, methyl, ethyl, n- or iso-propyl or n-, sec- or tert-butyl or straight chain or branched pentyl, hexyl.

Lower alkenyl is, for example, vinyl, allyl, 2-or 3-butenyl, isobutenyl or n-penta-2,4-dienyl.

Lower alkynyl is, for example, 1- or 2-propynyl.

Monocycloalkyl is, for example, cyclopropyl, dimethylcyclopropyl, cyclobutyl, cyclopentyl or cyclohexyl.

Bicycloalkyl is, for example, bornyl or norbornyl.

Cycloalkenyl is, for example, cyclopentadienyl or cyclohexenyl.

Heterocycloalkyl preferably contains 4 or 5 carbon atoms and one or two hetero atoms from the group O, S and N. Examples are the substituents derived from oxirane, azirine, 1,2-oxathiolane, pyrazoline, pyrrolidine, piperidine, piperazine, morpholine, tetrahydrofuran or tetrahydrothiophene.

Aryl is, for example, mono-, bi- or tri-cyclic, for example phenyl, naphthyl, indenyl, azulenyl or anthryl.

Heteroaryl is preferably monocyclic or condensed with a further heterocycle or with an aryl radical, for example phenyl, and preferably contains one or two, and in the case of nitrogen up to four, hetero atoms from the group O, S and N. Suitable substituents are derived from furan, thiophene, pyrrole, pyridine, bipyridine, picolylimine, y-pyran, y-thiopyran, phenanthroline, pyrimidine, bipyrimidine, pyrazine, indole, coumarone, thionaphthene, carbazole, dibenzofuran, dibenzothiophene, pyrazole, imidazole, benzimidazole, oxazole, thiazole, dithiazole, isoxazole, isothiazole, quinoline, isoquinoline, acridine, chromene, phenazine, phenoxazine, phenothiazine, triazine, thianthrene, purine or tetrazole.

Aralkyl preferably contains from 7 to 12 carbon atoms, for example, benzyl, 1- or 2-phenethyl or cinnamyl.

Heteroarylalkyl preferably consists of the mentioned heterocycles, which substitute, for example, C₁-C₄alkyl radicals, where possible in the terminal position, but also in the adjacent position (1-position) or in the alpha-position (2-position), depending upon the length of the carbon chain.

“Amino protecting group” refers to a chemical group that exhibits the following characteristics: (1) reacts selectively with the desired amino in good yield to give a protected substrate that is stable to the projected reactions for which protection is desired; 2) is selectively removable from the protected substrate to yield the desired functionality; and 3) is removable in good yield by reagents compatible with the other functional group(s) generated in such protected reactions. Examples of amino protecting groups can be found in Greene et al. (1991) Protective Groups in Organic Synthesis, 2^{nd} Ed. (John Wiley & Sons, Inc., New York).

In accordance with this invention, any conventional amino protecting group that can be removed by hydrogenolysis or hydrolysis can be utilized. Among the preferred amino protecting which can be utilized in accordance with this invention are trityl, benzyl, o-nitro benzyl, aromatic urethane-type protecting groups, such as benzyloxycarbonyl (Z) and substituted benzyloxycarbonyl, such as p-chlorobenzyloxycarbonyl, p-nitrobenzyloxycarbonyl, p-bromobenzyl-oxycarbonyl, p-biphenyl-isopropyloxycarbonyl, 9-fluorenylmethyl-oxycarbonyl (Fmoc) and p-methoxybenzyloxycarbonyl (Moz); aliphatic urethane-type protecting groups, such as t-butyloxycarbonyl (Boc), diisopropylmethyloxycarbonyl, isopropyloxycarbonyl, and allyloxycarbonyl. Boc is most preferred for alpha amino protection.

In accordance with this invention, R₁₃ can be any conventional leaving group. These leaving groups include halogen, such as chlorine and bromine, N-succinimidyloxy, sulfo-N-succinimidyloxy, 1-benzotriazolyloxy, 1-imidazolyl, p-nitrophenyloxy, 2,3,4-trichlorophenyloxy, pentachlorophenyloxy, pentafluorophenyloxy, N-phthalimidyloxy, N-tetrahydrophthalimide, N-glutarimide, 1-hydroxypiperidine, 5-chloro-8-hydroxy-quinoline, N-norbornene-2,3-dicarboximide, hydroxy-7-azabenzotriazole, mesyloxy, and tosyloxy with halogen or mesyloxy being preferred.

Combinatorial library synthesis is typically performed on a solid support. See, for example, Lam et. al. (1991) Nature 354:82-84; Houghton et al. (1991) Nature 354:84-86. A large number of beads or particles are suspended in a suitable carrier (such as a solvent) in a parent container. The beads, for example, are provided with a functionalized point of attachment for a chemical module. The beads are then divided and placed in various separate reaction vessels. The first chemical module is attached to the bead, providing a variety of differently substituted solid supports. Where the first chemical module includes 3 different members, the resulting substituted beads can be represented as A₁, A₂, and A₃.

The beads are washed to remove excess reagents and subsequently remixed in the parent container. This bead mixture is again divided and placed into various separate reaction vessels. The second chemical module is coupled to the first chemical module. Where the second chemical module includes 3 different members, B₁, B₂, and B₃, 9 differently substituted beads result: A₁-B₁, A₁-B₂, A₁-B₃, A₂-B₁, A₂-B₂, A₂-B₃, A₃-B₁, A₃-B₂, and A₃-B₃. Each bead will have only a single type of molecule attached to its surface.

The remixing/redivision synthetic process can be repeated until each of the different chemical modules has been incorporated into the molecule attached to the solid support. Through this method, large numbers of individual compounds can be rapidly and efficiently synthesized. For instance, where there are 4 different chemical modules, and where each chemical module contains 20 members, 160,000 beads of different molecular substitution can be produced.

When combinatorial library synthesis is performed manually, a scientist would perform the various chemical manipulations. For the construction of a combinatorial library through an automated process, the various chemical manipulations will typically be performed robotically. For example, see U.S. Pat. No. 5,463,564.

The synthesis of a 3-aryl, 2-carboxamide indole compound library can be performed on a solid support. “Solid support” includes an insoluble substrate that has been appropriately derivatized such that a chemical molecule can be attached to the surface of the substrate through standard chemical methods. Solid supports include, but are not limited to, beads and particles, such as peptide synthesis resins. For example, see Merrifield (1963) J. Am. Chem. Soc. 85:2149-2154; U.S. Pat. No. 4,631,211; and Geysen et al. (1984) Proc. Natl. Acad. Sci. USA 81:3998-4002.

Solid supports can consist of many materials, limited primarily by the capacity of the material to be functionalized through synthetic methods. Examples of such materials include, but are not limited to, polymers, plastics, resins polysaccharides, silicon or silica based materials, carbon, metals, inorganic glasses and membranes. Preferred resins include Sasrin resin (a polystyrene resin available from Bachem Bioscience, Switzerland), and TentaGel S AC, TentaGel PHB, or TentaGel S NH₂ resin polystyrene-polyethylene glycol copolymer resins available from Rapp Polymere, Tubingen, Germany).

The solid support can be purchased with suitable functionality already present such that a chemical module can be attached to the support surface (e.g., Novabiochem, Argonaut ArgoGel, Bachem Bioscience, Rapp Polymere). Alternatively, the solid support can be chemically modified such that a chemical module can be attached to the support surface. Grant (1992) Synthetic Peptides. A User's Guide, W. H Freeman and Co; Hermkens et al. (1996) Tetrahedron 52:4527-4554. The choice of functionality used for attaching a molecule to the solid support will depend on the nature of the compound to be synthesized and the type of solid support. Examples of functionality present on the solid support that can be used to attach a chemical module, include, but are not limited to, alkyl or aryl halides, aldehydes, alcohols, ketones, amines sulfides, carboxyl groups, aldehyde groups, and sulfonyl groups.

Preferably, the functional group on the solid support that permits the attachment of a chemical module will be an alcohol, an amine, an aldehyde, or a diol group Gordon et al. (1994) J. Med. Chem. 37:1385-1401; Hermkens et al. (1996) Tetrahedron 52:4527-4554.

Preferably, the reaction used to attach the chemical module to the solid support will be a reductive amination of a primary amine to aldehyde-containing solid phase polymer resin.

For the attachment of certain chemical modules to the solid support, masking of functionality that is not involved in the attachment process, but that is incompatible with the mode of attachment, may be necessary. A non-limiting example of this type of process is the esterification of an alcohol functionalized solid support, using a hydroxyl-substituted carboxylic acid as the coupling partner. Prior to the reductive animation reaction, the hydroxyl group of the carboxylic acid would be “protected” through alkylation, silylation, acetylation, or through some other standard method. Strategies for the use of masking or protecting groups have been well-described in the art, such as in Green (1985) Protecting Groups in Organic Synthesis; Wiley.

A general synthetic strategy for the construction of fused 3-aryl-2-carboxamido N₁-substituted pyroles containing libraries is shown in FIG. 1. This route employs the immobilization of a primary amine derivative to aldehyde containing solid phase resin.

To construct a 3-aryl-2-carboxamido N₁-substituted pyroles library through the immobilized amine derivative route, a chemical module containing a terminal amine, or protected terminal amine is attached to a solid support containing functionalized resin. Where the terminal amine of the chemical molecule is protected, the synthetic route proceeds through the deprotection of the terminal amine.

A solid support bound through a functionalized resin to a fused 3-aryl-2-carboxamido N₁-substituted pyrroles library can be recovered through conventional methods such as filtration or centrifugation. Confirmation that the solid support contains the desired fused 3-aryl-2-carboxamido N₁-substituted pyroles compound can be accomplished by cleaving the fused 3-aryl-2-carboxamido N₁-substituted pyroles from a small portion of the solid support, and then subjecting the cleaved product to conventional analysis. Examples of commonly used analytical methods include, but are not limited to, nuclear magnetic resonance spectroscopy and high performance liquid chromatography.

In one embodiment of the invention, the fused 3-aryl-2-carboxamido N₁-substituted pyroles library is bound to a solid support. In another embodiment of the invention, the fused 3-aryl-2-carboxamido N₁-substituted pyroles is cleaved from the solid support to produce soluble fused 3-aryl-2-carboxamido N₁-substituted pyroles libraries. Soluble libraries can be advantageous for a variety of purposes, including assaying the biological activity of compounds and performing structural analysis of compounds.

The cleavage of compounds from a solid support to produce a soluble chemical library can be accomplished using a variety of methods. For example, a compound can be photolytically cleaved from a solid support (Wang et al. (1976) J. Org. Chem 41:3258; Rich et al. (1975) J. Am. Chem. Soc. 97:1575-1579). Preferably, the cleavage of compounds from a solid support to produce a soluble chemical library is accomplished using hydrolytic conditions, such as through the addition of dilute trifluoroacetic acid.

The present invention is directed toward the generation of fused 3-aryl-2-carboxamido N₁-substituted pyroles libraries. These libraries are used to select one or more fused 3-aryl-2-carboxamido N₁-substituted pyroles species that demonstrate a specific interaction with a targeted cellular ligand including, but not limited to, enzymes or receptors. A cellular ligand is targeted when it is believed that the ligand is of importance in the modulation of a disease. Examples of disease states for which fused 3-aryl-2-carboxamido N₁-substituted pyroles libraries can be screened include, but are not limited to, inflammation, infection, hypertension, CNS disorders, and cardiovascular disorders.

Several methods have been developed in recent years to screen libraries of compounds to identify the compounds having the desired characteristics. Typically, where a compound exhibits a dissociation constant of 10⁻⁶ or less when combined with the targeted enzyme or receptor, the compound is thought to demonstrate a specific interaction with the enzyme or receptor. Methods for isolating library compound species that demonstrate desirable affinity for a receptor or enzyme are well-known in the art. For example, an enzyme solution may be mixed with a solution of the compounds of a particular combinatorial library under conditions favorable to enzyme-ligand binding. See Bush et al. (1993) Antimicrobial Agents and Chemotherapy 37:851-858, and Daub et al. (1989) Biochemistry 27:3701-3708. Specific binding of library compounds to the enzyme may be detected by any of the numerous enzyme inhibition assays which are well known in the art. Compounds which are bound to the enzyme may be readily separated from compounds which remain free in solution by applying the solution to a Sephadex G-25 gel filtration column. Free enzyme and enzyme-ligand complexes will pass through the column quickly, while free library compounds will be retarded in their progress through the column. The mixture of enzyme-ligand complex and free enzyme can then be treated with a powerful denaturing agent, such as guanidinium hydrochloride or urea, to cause release of the ligand from the enzyme. The solution can then be injected onto an HPLC column (for example, a Vydac C-4 reverse-phase column, eluted with a gradient of water and acetonitrile ranging from 0% acetonitrilc to 80% acetonitrile). Diode array detection can provide discrimination of the compounds of the combinatorial library from the enzyme. The compound peaks can then be collected and subjected to mass spectrometry for identification.

An alternate manner of identifying compounds that inhibit an enzyme is to divide the library into separate sub-libraries where one step in the synthesis is unique to each sub-library. To generate a combinatorial library, reactants are mixed together during a step to generate a wide mixture of compounds. At a certain step in the synthesis, however, the resin bearing the synthetic intermediates can be divided into several portions, with each portion then undergoing a unique transformation. The resin portions are then separately subjected to the rest of the synthetic steps in the combinatorial synthetic method. Each individual resin portion thus constitutes a separate sub-library. When testing the compounds, if a given sub-library shows more activity than the other sub-libraries, the unique step of that sub-library may then be held fixed. The sub-library then becomes the new library, with that step fixed, and forms the basis for another round of sub-library synthesis, where a different step in the synthesis is optimized. This procedure can be executed at each step until a final compound is arrived at. The aforementioned method is the generalization of the method described in Geysen, WO 86/00991, for determining peptide “mimotopes,” to the synthetic method of this invention.

Finding a compound that inhibits an enzyme is most readily performed with free compound in solution. The compounds can also be screened while still bound to the resin used for synthesis; in some applications, this may be the preferable mode of finding compounds with the desired characteristics. For example, if a compound that binds to a specific antibody is desired, the resin-bound library of compounds may be contacted with an antibody solution under conditions favoring a stable antibody-compound-resin complex. A fluorescently labeled second antibody that binds to the constant region of the first antibody may then be contacted with the antibody-compound-resin complex. This will allow identification of a specific bead as carrying the compound recognized by the first antibody binding site. The bead can then be physically removed from the resin mixture and subjected to mass spectral analysis. If the synthesis has been conducted in a manner such that only one compound is likely to be synthesized on a particular bead, then the binding compound has been identified. If the synthesis has been carried out so that many compounds are present on a single bead, the information derived from analysis can be utilized to narrow the synthetic choices for the next round of synthesis and identification.

The enzyme, antibody, or receptor target need not be in solution either. Antibody or enzyme may be immobilized on a column. The library of compounds may then pass over the column, resulting in the retention of strongly binding compounds on the column after weaker-binding and non-binding compounds are washed away. The column can then be washed under conditions that dissociate protein ligand binding, which will remove the compounds retained in the initial step. These compounds can then be analyzed, and synthesized separately in quantity for further testing. Similarly, cells bearing surface receptors can be expressed on a cell surface may be contacted with a solution of library compounds. The cells bearing bound compounds can be readily separated from the solution containing non-binding compounds. The cells can then be washed with a solution which will dissociate the bound ligand from the cell surface receptor. Again, the cells can be separated from the solution.

In accordance with this invention, the solid support is bound via an amine linkage to the molecule and contains a functional group reactive with an amine: 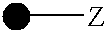
wherein ● is the solid support Z is a functional group reactive with an amine.

Therefore in accordance with this invention it is best to provide a solid support which can be functionalized to a group which will react with the amine of the formula:
R₂NH₂ VII
wherein R₂ is as above
   to immobilize a primary amine on a solid support to produce a compound of the formula: 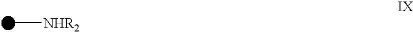
wherein ● is the solid support, and R₂ is as above.

In accordance with a preferred embodiment of this invention, the solid support contains an aldehyde group so that it is easily animated to produce the compound to formula IX. Any conventional means of reductive animation can be used to react the solid support containing a reactive functional aldehyde group with the amine of formula VII to produce the compound to formula IX. Particularly those of reductive animation reactions are carried out utilizing an alkali metal brohydride reducing agent such as sodium brohydride or sodium actoxy brohydride. In carrying this reaction, any of the conventional conditions in reductive animation can be utilized. Generally is preferred to carry out this reaction in a polar organic solvent. Any conventional inert polar organic inert solvent, such as methylene chloride, ethylene chloride, etc. can be utilized. In carrying out this reaction to produce the compound of formula IX temperature and pressure are not critical in this reaction can be carried out at room temperature and atmospheric pressure.

In accordance with this invention, the compound of formula IX is converted to the compound of formula I above, where R₁ is other than hydrogen by the following procedure:
coupling said immobilized amine of formula IX to an organic acid of the formula: 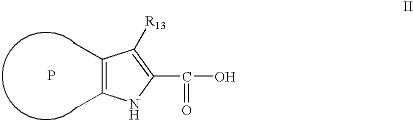
wherein P, is as above, and R₁₃ is a leaving group,
   to produce an immobilized amide of the formula: 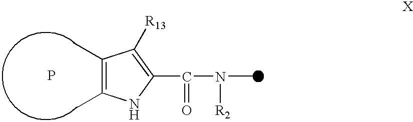
wherein ●, P, R₂, and R₁₃ are as above,
   reacting the compound of formula X with a halide of the formula
   R₁ halo XI
wherein R₁ is as above other than hydrogen and halo is a halide
   to produce a compound of the formula 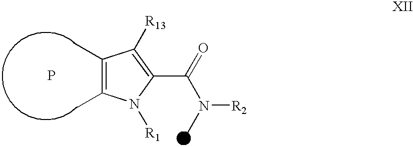
   wherein ●, P, R₁, R₂, and R₁₃ are as above and R₁ is other than hydrogen,
   reacting said indole of formula XI with a boronic acid of the formula 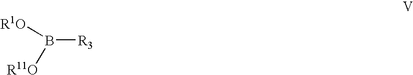
   wherein R₃ is as above R¹ and R¹¹ are individually lower alkyl or taken together form a lower alkylene bridge between their attached oxygen atoms,
   to produce an immobilized compound of the formula I where R₁ is other than hydrogen of the formula: 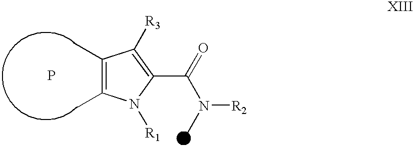
wherein ●, P, R₂, and R₃ are as above and R₁ is as above other than hydrogen,
   and cleaving by hydrolysis said immobilized compound of formula XIII from said solid support to produce the compounds of formula I.

The coupling reaction of the amine of formula IX with the organic acid of formula II is carried out to produce the immobilized amide of formula X by utilizing any conventional method of reacting an organic acid with an amine to produce amide. In carrying out this reaction, any of the conditions conventional in peptide synthesis can be utilized. Generally this coupling reaction takes place in the presence of an organic base, such as a tertiary alkyl amine. Any of the conventional conditions utilized in peptide synthesis can be utilized to condense the compound of formula II with the compound of formula IX to produce the amide of formula X.

In the next step, the amino group at the 1-position on the indole ring of the compound of formula X is reacted with the halide of formula XI to produce the compound of formula XII. Any conventional method of condensing an amine with a halide so as to convert a secondary amine to a tertiary amine can be utilized in this synthesis. The reaction of the halide of formula XI is used where one wants to prepare compounds of formula X where R₁ is other than hydrogen and produce the compounds of formulae XII and I where at the 1-position on the indole ring, R₁ is other than hydrogen. On the other hand if it is desired to produce a compound of formula I where at the 1-position on the indole ring R₁ is hydrogen, then it is necessary to protect the nitrogen with a suitable amino protecting group. Any of the conventional amino protecting groups can be utilized and any method conventional in protecting a secondary amine with a protecting group such as BOC can be utilized. In accordance with this invention any conventional amino protecting group can be utilized for this purpose of producing a compound to formulae I and XII where R₁ is hydrogen.

In the next step of this synthesis, the compound of formula XII, where either R₁ is not hydrogen or where R₁ is replaced by a conventional amino protecting group, is reacted with a boronic acid of the formula V to produce the compound of formula XIII. In the case where, at the 1-position on the indole ring, R₁ is replaced by an amino protecting group in the compound of formula XII, this amino protecting group will also be at the 1-position in the compound of formula XIII. In accordance with this invention, the amino group at the one position of indole ring in the compound of formula XII should not contain a hydrogen substituent in this reaction. The reaction of the compound of the formula XII with a compound of formula IV is carried out by utilizing a Suzuki coupling reactions, such as disclosed by S. S. Bhawgwat et al. Tetrahedron Lett. 1994, 35 p. 1847-1850. In carrying out this reaction, any of the conditions conventional in a Suzuki reaction can be utilized. Generally, these reactions are carried out in the presence of a metal catalyst such as a palladium catalyst utilizing any invention inert solvent. Among the preferred solvents are the polar aprotic solvents Any conventional inert polar aprotic solvents can be utilized in carrying out this invention. Suitable solvents are customary, especially higher-boiling, solvents, for example non-polar aprotic solvents, e.g., xylene or toluene, or polar aprotic solvents, e.g., dimethoxyethane. In this manner, by either placing a substituent on or protecting the nitrogen atom at the 1-position on the indole ring, one can easily produce the compound of formula XIII by carried out utilizing the Suzuki reaction with the boronic acid of formula VI.

In the coupling reaction involving the compound of formula V with the compound of formula XII, the leaving group is eliminated. In the coupling reaction, especially in the Suzuki reaction, suitable leaving groups, are for example, halogen, e.g., chlorine, bromine or iodine, or an organosulfonyl radical, for example mesyl, p-toluenesulfonyl(tosyl)bmm or trifluoromethanesulfonate. Iodine is the preferred leaving group in the Suzuki type reactions. Coupling reactions of the Suzuki type occur with excellent yield and high purity.

A preferred embodiment of the Suzuki type reaction utilizes a palladium-catalyst-and a substituted aryl chloride deactivated by means of electron-rich or electron-repelling groups. The “catalytic amounts” of the palladium type catalyst preferably denotes an amount of from approximately 0.0001 to 5.0 mol %, especially from 0.001 to 1.0 mol %, based on the amount of the substrate used. The molar ratio of the reaction partners of the Suzuki coupling reaction of the boronic acid derivative of formula V to the compound of formula XII is generally in the range of from 1:1 to 1:10, a ratio in the range of from 1:1 to 1:2 being preferred. In carrying out this reaction temperature and pressure are not critical however it is preferred that this reaction take place with cooling up to the boiling temperature of the solvent, especially from room temperature to the boiling temperature of the solvent (reflux conditions). Working up and isolation of the obtainable reaction product are effected in a manner known in the art using customary purification methods, for example, removal of the solvent and subsequent separation methods, e.g., fine distillation, re-crystallization, preparative thin-layer chromatography, column chromatography, preparative gas chromatography, etc.

In accordance with this procedure compounds of formula III, IV and XIII can be cleaved from the resin support by any of the methods above. Generally, it is preferred to cleave these compounds by acid hydrolysis utilizing a strong acid or trifluoroacetic acid, or mineral acids. Any conventional method of cleaving amides s from the solid support such as used in solid peptides synthesis can be employed in the process of this invention. In this manner, the compounds will be cleaved from their solid support and where the nitrogen at the 1-position in the indole ring contains an amino protecting group, this amino protecting group will be hydrolyzed under this acid hydrolysis to produce the compound of formula I where R₁ is hydrogen. If one desires to produce the compound of formula III where R₁₄ is hydrogen, then an amino protecting group which can be removed hydrogenolysis is chosen to be the protecting group at the 1-position on the indole ring. By removing this amino protecting group by hydrogenolysis, the solid support will remain connected to the molecule. Hence, removal of the amino protecting group can be accomplished without cleaving the solid support. In this manner, the compound of formula III, where R₁₄ is hydrogen is produced.

In accordance with this invention, the organic acid of formula II is prepared from the compound of the formula 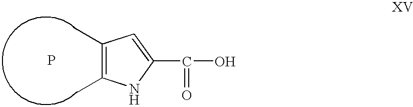
by placing a leaving group such as disclosed above and the 3-position of the indole ring. The 3-position is particularly reactive to the placement of a leaving group as a substituent at this position. The preferred leaving group is halide, particularly an iodo substituent.

In carry out this reaction, the compound of formula XV is treated with a halogenating agent, such as a halogen in a solvent, such as iodine dissolved in dimethyl-formamide, or a halosuccinimide in a conventional solvent medium. Any of the conditions conventionally utilized in halogenating with these halogenating agents can be utilized to carry out this reaction and produce a halo substituent at the 3-position on the indole ring. These halogen aiding agents will selectively halogenate the 3-position on the indole ring without affecting the other positions. The reaction whereby halogenating agents such as iodine are used to halogenate the compound of formula XV, can be carried out utilizing the same procedure as disclosed by Sakmoto et al. in Chem. Pharm. Bul., 1988, 36, 2248-2252. In addition any of the conventional well known procedures for providing other leaving groups such as mesyloxy or tosyloxy can be utilized to produce a leaving group at the 3-position of the indole ring on the compound of formula XV.

### EXAMPLES

The following examples are provided to illustrate, but not limit, the invention. Although the forgoing invention has been described in some detail by way of illustration and example for purposes of clarity and understanding, it will be apparent to those skilled in the art that certain changes and modification may be practical. Therefore, the description and examples should not be construed as limiting the scope of the invention, which are delineated by the appended claims. Further, the methods for synthesis of single compounds are directly amenable to the synthesis of small molecule compound libraries using split-and-pool techniques, which are known in the art.

### General Methods

Reagents were purchased from Aldrich, Sigma, Bachem Biosciences, Advanced ChemTech, Lancaster and Argonaut Argogel and used without further purification. Washing resins, either free flowing or in devices, for effecting solvent permeable resin segregation appropriate for split and mix combinatorial synthesis involves the addition of a stated solvent and agitation of the solid phase in that solvent for at least 3 minutes before the solvent is then filtered away from the solid phase polymer. This constitutes washing one time; solid phase polymers are routine washed several times in a series of solvents. After cleavage of organic products from the solid phase, concentration of solutions was performed by reduced pressure rotary evaporation, or using the Savant SpeedVac and Genevac rotary evaporator instruments. NMR (nuclear magnetic resonance) spectra were recorded on a Bruker 300 Mhz instrument with CDCl₃ as solvent unless noted. ¹H NMR data are reported as follows: chemical shifts relative to tetramethylsilane (0.00 ppm), multiplicity (s=singlet, d=doublet, dd=doublet of doublets, t=triplet, m=multiplet), coupling, and integration. Assignment of protons was aided by decoupling experiments. LC/MS (liquid chromatography/mass spectroscopy) spectra were recorded using the following system. For measurement of mass spectra, the system was configured with a Micromass Platform II: API Ionization in positive electrospray (mass range: 150-1200 amu). The simultaneous chromatographic separation was achieved with the following HPLC system: Column, ES Industries Chromegabond WR C-18 3u 120 Å (3.2×30 mm) Cartridge; Mobile Phase A: Water (0.02% TFA) and Phase B: Acetonitrile (0.02% TFA); gradient 10% B to 90% B in 3 minutes; equilibration time, 1 minute; flow rate of 2 ml/minute.

The following abbreviations are used in the description of experimental procedures: eq for equivalent; DMF for dimethyl-formamide, NaHCO₃ for sodium bicarbonate, HATU for O-(7-azabenzotriazol-1-yl)-1,1,3,3-tetramethyluronium hexafluorophosphate, HBTU for O-(benzotriazol-1-yl)-1, 1,3,3-tetramethyluronium hexafluorophosphate, DIPEA for diisopropyl-ethylamine, DME for dimethylethyleneglycol, CH₃CN for acetonitrile, DCM or CH₂Cl₂ for dichloromethane, CH₃OH for methyl alcohol, ClCH₂CH₂Cl for dichloroethane, TFA for trifluoroacetic acid, Boc for bis-ter-butyloxycarbonyl, DME for dimethoxyethane, and FMPB for formylmethoxyphenoxybutyric acid amide derivatived polystyrene.

General procedure for synthesis of 3-iodo-1H-indole-2-carboxylic acid:
1.0 mmol of 1H-Indole-2-carboxylic acid and 3.8 mmol of KOH were dissolved in 7 ml of water. 1 mmol of I₂ was dissolved in 1.1 ml of DMF. The I₂ solution was added to the aqueous solution drop wise. The result solution was stirred for another 30 minutes. The reaction was monitored by HPLC. The solution was acidified by 1N hydrochloric acid to pH 4 to 5. 3-iodo-1H-indole-2-carboxylic acid was filtered and washed by more water.

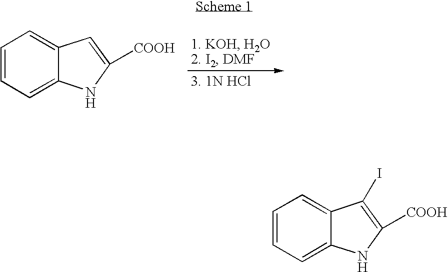

Alternative procedure for synthesis of 3-iodo-1H-indole-2-carboxylic acid:

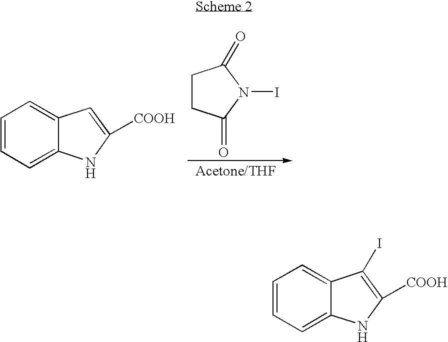

This procedure was carried out as disclosed by Sakamoto et al. (Sakamoto, T.; Nagano, T.; Kondo, Y.; Yamanaka, H. Chem. Pharm. Bull., 1988, 36, 2248-2252). 1.0 mmol of 1H-Indole-2-carboxylic acid was dissolved in 10 ml of acetone. 1.0 mmol of N-iodosuccinimide (NIS) was dissolved in 2 ml of acetone. The NIS solution was added to the solution of 1H-Indole-2-carboxylic acid dropwise. The reaction solution was stirred for another hour. At this time, the reaction mixture was concentrated under reduced pressure. The resulting solid was washed by water three times and filtered. The final product was dried under the vacuum.

Method A: General Procedures for Solid Phase Preparations of 3-aryl-1H-indole-2-carboxamides.
a) Reductive amination: FMPB resin (Argonaut Inc.) (10 g, 1.1 mmol/g) was mixed with benzyl amine (88 mmol) in 100 ml of methylene chloride/acetic acid (vol/vol:2/1) for 10 minutes. Sodium triacetoxylboron hydride (Aldrich, 55 mmol) was added to the solution. The suspension was stirred for 14 hours at room temperature. At this time, the resin was filtered and washed with 0.5N NaHCO₃/MeOH (1:5) three times. Then the resin was further washed with DMF four times, with methanol four times, methylene chloride four times and hexanes four times. The resin washed in this manner was dried then under the vacuum. Four other primary amines were similarly loaded to separate batches of FMPB resin. The resin was loaded into resin segregation devices. Each can was approximately containing 88 micromoles of resin bound amine.
b) Acylation: To a suspension of 100 resin segregation devices (88 μmol equivalent) resin segregation devices, 8.8 mmol in total) in 120 ml of DMF were added 3-iodo-1H-indole-2-carboxylic acid (12.63 g, 44 mmol), HATU (16.72 g, 44 mmol), and di-isopropyl ethyl amine (44 mmol). The suspension was shaken overnight at room temperature under an atmosphere of argon. The solvent was filtered and the Resin segregation devices were washed with DMF four times, with methanol four times, methylene chloride four times and hexanes four times. The Resin segregation devices were dried under the vacuum over night at room temperature. Fifteen other different substituted 3-iodo indole-2-carboxylic acids were similarly loaded to separate batches of amine resin.
c) BOC protection: To the 500 resin segregation devices (88 μmol equivalent) Resin segregation device, 44 mmol in total) in 500 ml of DMF was added di-tert-butyl dicarbonate (50.5 ml, 0.22 mol), DMAP(5.38 g, 44 mmol), and triethylamine (62 ml, 0.44 mol). The suspension was shaken overnight under an atmosphere of argon. The solvent was filtered and the Resin segregation devices were washed with DMF four times, with methanol four times, methylene chloride four times and hexanes four times. The resin segregation devices were dried under the vacuum over night at room temperature and sorted.
d) Aryl coupling: To 10 resin segregation devices (0.88 mmol total equivalence) in 10 ml of DME was added tetrakis(triphenylphosphine)palladium (0) (0.01 g, 0.132 mmol) and shaken for 15 minutes. Phenyl boronic acid (4.4 mmol) and Na₂CO₃ (2M, 2 ml, 4.9 mmol) were added to the moisture. The suspension was heated at 90° C. for 14 hours under an argon atmosphere. The solvent was filtered off and the Resin segregation devices were washed with DMF four times, with methanol four times, methylene chloride four times and hexanes four times. The Resin segregation devices were dried under the vacuum over night at room temperature.
e) Cleavage: The Resin segregation devices were sorted into single cleavage wells and treated with TFA in DCM (vol/vol 1:1) with continuous vibration as a means of agitation at room temperature for 2 hours. The solution was drained into bar coded 2 dram vials and the resin was rinsed with one 1 mL DCM. The TFA/DCM solvents were removed under reduced pressure on a Savant SpeedVac or a Genevac rotary evaporator to give crude 3-phenyl-1H-indole-2-carboxylic acid benzylamides.

The compound shown in Example 1 is a typical compounds obtained via Method A.

### Example 1

### 5-chloro-3-phenyl-1H-indole-2-carboxylic Acid Benzylamide

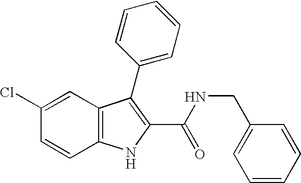

¹H-NMR (CDCl₃): 10.38(s, 1H), 7.00-7.60 (m, 13H), 6.36(s, 1H), 4.49(d; J=5.6 Hz, 2H). LCMS(10-90% acetonitrile): C₂₂H₁₇ClN₂O=360.00, 360.11, 2.86 min, 100%.

Method B: General Procedures for Solid Phase Preparations of 1-substituted-3-aryl-1H-indole-2-carboxylic acid amides (FIG. 3).
a) Reductive amination: Amines were loaded to FMPB resin as described in Method A, a).
b) Acylation: 3-Iodo-1H-Indole-2-carboxylic acids loaded to above amine resin as described in Method A, b).
c) N¹-alkylation: To a suspension of 500 resin segregation devices (88 μmol equivalent/Resin segregation device, 44 mmol total for 500 resin segregation devices) in 500 ml of DMF was added NaH (60% dispersion in mineral oil, 14.0 g, 0.35 mol), The suspension was shaken 30 min at RT. At that time, benzyl bromide (4.50 g, 0.26 mmol) was added. The reaction mixture was shaken overnight under an atmosphere of argon. The solvent was filtered and the Resin segregation devices were washed with DMF four times, with methanol four times, methylene chloride four times and hexanes four times. The Resin segregation devices were dried under the vacuum over night at room temperature and sorted.
d) Aryl coupling: Aryl coupling reactions were carried out as described in Method A, d).
e) Cleavage: Cleavage reactions were carried out as described in Method A, e).

The compound shown in Example 2 is a typical compounds obtained via Method B.

### Example 2

benzyl-2-(Benzylaminocarbonyl)-5-chloro-3-phenylindole

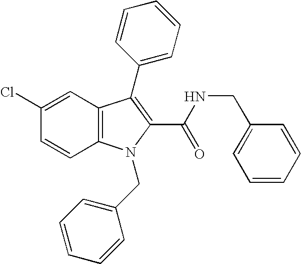

¹H-NMR (CDCl₃): 6.91-7.60 (m, 16H), 6.90-6.79(m, 2H), 5.85-5.80(m, 1H), 5.77(s, 2H), 4.33(d, J=5.8 Hz, 2H). LCMS(25-90% acetonitrile): C₂₉H₂₃ClN₂O=450, 451.22, 2.86 min, 100%.

Method C: General Procedures for Solid Phase Preparations of 1-unsubstituted-3-aryl-1H-indole-2-carboxylic acid amides (FIG. 4).
a) Load of 3-iodo-1H-indole-2-carboxylic acid to Wang Resin: To a suspension of 100 Resin segregation devices each containing Wang Resin HL (IRORI Unisphere 200, 88 μmol equivalent/Resin segregation device, 8.8 mmol in total) in 120 ml of DMF were added 3-iodo-1H-indole-2-carboxylic acid (44 mmol), HATU (16.72 g, 44 mmol), i-isopropyl ethyl amine (44 mmol). The suspension was shaken overnight at room temperature under an atmosphere of argon. The solvent was filtered and the Resin segregation devices were washed with DMF four times, with methanol four times, methylene chloride four times and hexanes four times. The Resin segregation devices were dried under the vacuum over night at room temperature.
b) BOC protection: The Resin segregation devices were suspended in 120 ml of DMF and BOC anhydride (50.5 ml, 0.22 mol), DMAP (5.38 g, 44 mmol), triethylamine (62 ml, 0.44 mol). The suspension was shaken overnight under an atmosphere of argon. The solvent was filtered and the Resin segregation devices were washed with DMF four times, with methanol four times, methylene chloride four times and hexanes four times. The Resin segregation devices were dried under the vacuum over night at room temperature.
c) Aryl coupling and cleavage: To 10 resin segregation devices (0.88 mmol total equivalence) in 10 ml of DME was added tetrakis(triphenylphosphine)palladium (0) (0.15 g, 0.132 mmol) and shaken for 15 minutes. Phenyl boronic acid (4.4 mmol) and 2M (aqueous) Na₂CO₃ (2 ml, 4.9 mmol) were added to the solution. The suspension was heated at 90° C. for 14 hours under an argon atmosphere. The solvent were filtered off and the Resin segregation devices were washed with DMF four times, with methanol four times, methylene chloride four times and hexanes four times. The Resin segregation devices were dried under the vacuum over night at room temperature.
d) Cleavage: The Resin segregation devices were sorted into single cleavage wells and taken into the cleavage using TFA in DCM (vol/vol 1:1) at room temperature for 2 hours. The solution was drained into tared, bar coded vials and the resin was rinsed with one 1 mL DCM. The solvents were removed under reduced pressure on a Savant SpeedVac or Genevac rotary evaporator instruments providing the crude 3-phenyl-1H-indole-2-carboxylic acid benzylamide.

1. A combinatorial library comprising a library containing a plurality of different compounds having the formula: 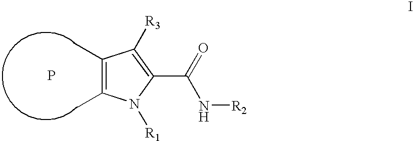
wherein P is a fused ring substituent, which ring substituent is an aromatic ring, a heteroaromatic ring or a cycloaliphatic ring which may be substituted or unsubstituted; R₁ and R₂ are individually hydrogen, lower alkyl containing from 1 to 7 carbon atoms, lower alkenyl containing from 2 to 7 carbon atoms, lower alkynyl containing from 3 to 7 carbon atoms, mono or bi-cycloaliphatic ring with each ring having from 3 to 7 carbon atoms, aryl containing from 1 to 3 fused aromatic rings each ring consisting of 6 carbon atoms, heterocyloaliphatic containing 1 to 2 fused rings with each ring containing from 3 to 6 carbon atoms with one or two hetero atoms selected from the group consisting of O, S and N, monocyclic or bicyclic heteroaryl rings each containing from 3 to 6 carbon atoms with 1 to 4 hetero atoms which can be N, S or O with the proviso that when the hetero atom is S or O, there are 1 or 2 hetero atoms in the ring and when the hetero atoms is N there are from 1 to 4 N atoms in the ring, and wherein the hetero ring in the heterocycloaliphatic ring or monocyclic or bicyclic heteroaryl rings can be condensed with an aryl, or cycloaliphatic ring and wherein any of the heteroaryl, aryl cycloaliphatic or heteroaliphatic rings in the cyloaliphatic aryl, heteroaryl or heteroaliphatic substituents may be connected to the formula I by a lower alkylene chain containing from 1 to 7 carbon atoms and R₃ is a ring substituent selected from the group consisting of an aromatic ring, a heteroaromatic ring and a cycloaliphatic ring which ring substituent may be substituted or unsubstituted.

2. The combinatorial library of claim 1 wherein when either P and R₃ are substituted ring substituents the substitution is by one or more radicals selected from the group consisting of amino, C₁-C₄ alkylamino, di-C₁-C₄ alkylamino, hydroxy, oxo, thio, nitro, carboxy, carbamoyl, sulfo, sulfamoyl, ammonio, amidino, cyano, formylamino, formamido, halogen, saturated or unsaturated aliphatic, cycloaliphatic or heterocycloalkyl; or wherein the heteroaromatic aromatic or cycloaliphatic rings which form P or R₃ may be substituted by condensing the rings with a further heteroaromatic, cycloaliphatic or aromatic ring which may be unsubstituted or substituted with one or more of said radicals.

3. The combinatorial library of claim 2 wherein the library contains at least 200 different compounds having the structure of formula I.

4. The combinatorial library of claim 3 wherein the library contains from about 500 to 10,000 different compounds having the structure of formula I.

5. The library of claim 3 wherein said library is randomized.

6. The combinatorial library of claim 1, wherein said library contains a plurality of different compounds having a formula from the group consisting: 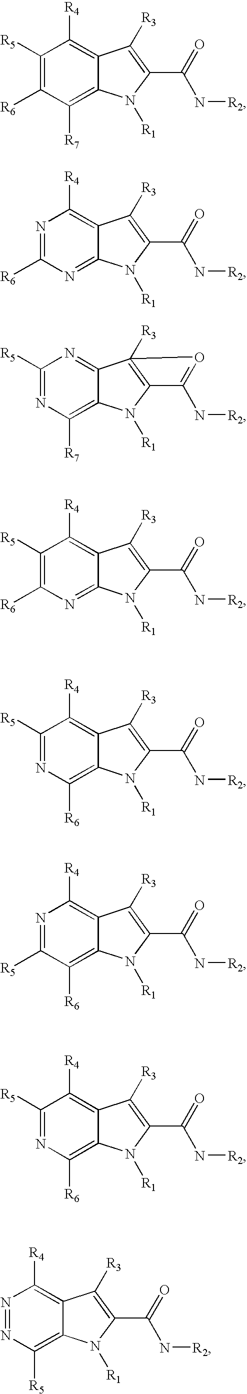 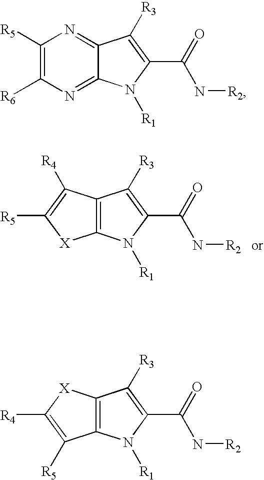
wherein R₁, R₂ and R₃ are as above; and R₄, R₅, R₆ and R₇ are individually selected from functional groups or derivatized functional groups consisting of amino, C₁-C₄alkylamino, di-C₁-C₄alkylamino, hydroxy, oxo, thio, nitro, carboxy, carbamoyl, sulfo, sulfamoyl, ammonio, amidino, cyano, formylamino, formamido, halogen, saturated or unsaturated, cycloalkyl, heterocycloalkyl, aryl, or heteroaromatic rings which may be condensed with aryl, heteroaromatic or heterocycloalkyl rings and X is O or S.

7. The combinatorial library of claim 6
wherein R₃ is 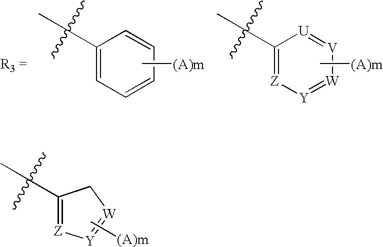
wherein m is an integer of from 1 to 3, A is R₄, R₅, R₆ and R₇ and U, V, W, Y and Z are individually —N—, —O—, —S— or —CH— with at least one of U, V, X or Y being —S—, —O— or —N—.

8. The combinatorial library comprising a library containing a plurality of different compounds having the formula immobilized on a solid support as follows: 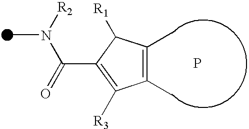
wherein ● is a solid support, wherein P is a fused ring substituent, which ring substituent is an aromatic ring, a heteroaromatic ring or a cycloaliphatic ring which may be substituted or unsubstituted; R₂ is a substituents selected from the group consisted of hydrogen, lower alkyl containing from 1 to 7 carbon atoms, lower alkenyl containing from 2 to 7 carbon atoms, lower alkynyl containing from 3 to 7 carbon atoms, mono and bicycloaliphatic ring with each ring having from 3 to 7 carbon atoms, aryl containing from 1 to 3 fused aromatic rings each ring consisting of 6 carbon atoms, heterocyloaliphatic containing 1 to 2 fused rings with each ring containing from 3 to 6 carbon atoms with one or two hetero atoms selected from the group consisting of O, S and N, monocyclic or bicyclic heteroaryl rings each containing from 3 to 6 carbon atoms with 1 to 4 hetero atoms which can be N, S or O with the proviso that when the hetero atom is S or O, there are 1 or 2 hetero atoms in the ring and when the hetero atoms is N, there are from 1 to 4 N atoms in the ring, and wherein the hetero ring in the heterocycloaliphatic ring or monocyclic or bicyclic heteroaryl rings can be condensed with an aryl, or cycloaliphatic ring and wherein any of the heteroaryl, aryl cycloaliphatic or heteroaliphatic rings in the cycloaliphatic aryl, heteroaryl or heteroaliphatic substituents may be connected to the formula I by a lower alkylene chain containing from 1 to 7 carbon atoms and R₃ is a ring substituent selected from the group consisting of an aromatic ring, a heteroaromatic ring and an cycloaliphatic ring which ring substituent may be substituted or unsubstituted and R₁ is an amino protecting group or anyone of the substituents given for R₂.

9. The combinatorial library of claim 8 wherein when either P and R₃ are substituted ring substituents, the substitution is by one or more radicals selected from the group consisting of amino, C₁-C₄ alkylamino, di-C₁-C₄ alkylamino, hydroxy, oxo, thio, nitro, carboxy, carbamoyl, sulfo, sulfamoyl, ammonio, amidino, cyano, formylamino, formamido, halogen, saturated or unsaturated aliphatic, cycloaliphatic or heterocycloalkyl; or wherein the heteroaromatic aromatic or cycloaliphatic rings which form P or R₃ maybe substituted by condensing the rings with a further heteroaromatic, cycloaliphatic, or aromatic ring which may be unsubstituted or substituted with one or more of said radicals.

10. The combinatorial library of claim 9, wherein said compounds which are immobilized to said solid support constitute a plurality of different compounds having a formula selected from the group consisting of: 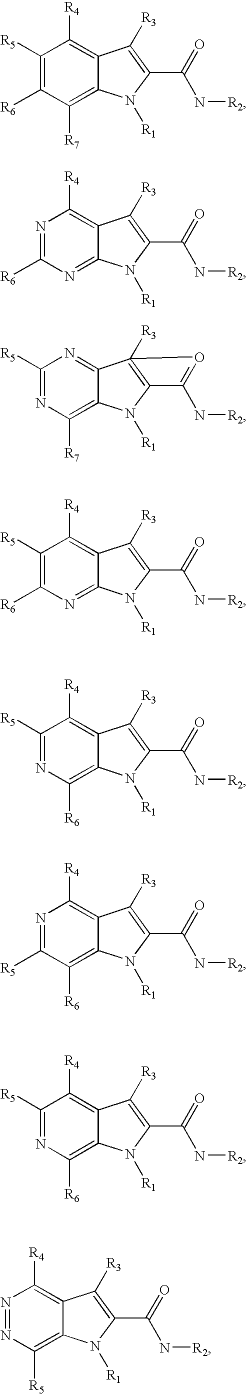 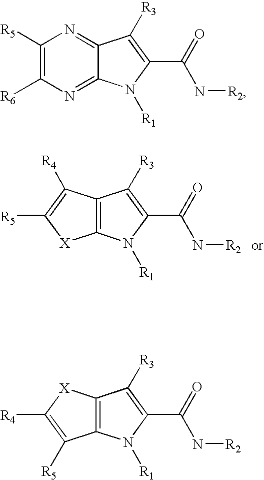
wherein R₁, R₂ and R₃ are as above; and R₄, R₅, R₆ and R₇ are individually selected from functional groups or derivatized functional groups consisting of amino, C₁-C₄alkylamino, di-C₁-C₄alkylamino, hydroxy, oxo, thio, nitro, carboxy, carbamoyl, sulfo, sulfamoyl, ammonio, amidino, cyano, formylamino, formamido, halogen, saturated or unsaturated, cycloalkyl, heterocycloalkyl, aryl, or heteroaromatic rings which may be condensed with aryl, heteroaromatic or heterocycloalkyl rings and X is O or S.

11. The combinatorial library of claim 10
wherein R₃ is 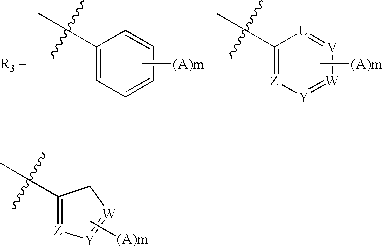
wherein m is an integer of from 1 to 3, A is R₄, R₅, R₆ and R₇ and U, V, W, Y and Z are individually —N—, —O—, —S— or —CH— with at least one of U, V, X or Y being —S—, —O— or —N—.

12. A method of preparing a combinatorial library containing a plurality of different compounds having the formula: 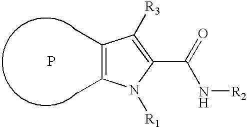
wherein P is a fused ring substituent, which ring substituent is an aromatic ring, a heteroaromatic ring or a cycloaliphatic ring which may be substituted or unsubstituted; R₁ and R₂ are individually hydrogen, lower alkyl containing from 1 to 7 carbon atoms, lower alkenyl containing from 2 to 7 carbon atoms, lower alkynyl containing from 3 to 7 carbon atoms, mono and bicycloaliphatic ring with each ring having from 3 to 7 carbon atoms, aryl containing from 1 to 3 fused aromatic rings each ring consisting of 6 carbon atoms, heterocyloaliphatic containing 1 to 2 fused rings with each ring containing from 3 to 6 carbon atoms with one or two hetero atoms selected from the group consisting of O, S and N, monocyclic or bicyclic heteroaryl rings each containing from 3 to 6 carbon atoms with 1 to 4 hetero atoms which can be N, S or O and when the hetero atom is S or O, there are 1 or 2 hetero atoms in the ring and when the hetero atoms is N, there are from 1 to 4 N atoms in the ring, and wherein the hetero ring in the heterocycloaliphatic ring or monocyclic or bicyclic heteroaryl rings can be condensed with an aryl, or cycloaliphatic ring and wherein any of the heteroaryl, aryl cycloaliphatic or heteroaliphatic rings in the cyloaliphatic aryl, heteroaryl or heteroaliphatic substituents may be connected to the formula I by a lower alkylene chain containing from 1 to 7 carbon atoms and with the proviso that R₁ is other than hydrogen; and R₃ is ring substituent selected from the group consisting of an aromatic ring, a heteroaromatic ring and a cycloaliphatic ring which ring substituent may be substituted or unsubstituted comprising:
a) immobilizing on a solid support an amine of the formula:
R₂NH₂
wherein R₂ is as above
to produce an immobilized amine of the formula: 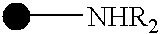
wherein ● is the solid support, and R₂ is as above
b) coupling said immobilized amine to an organic acid of the formula: 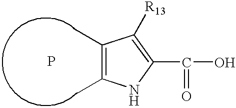
wherein P is as above, and R₁₃ is a leaving group,
to produce an immobilized amide of the formula: 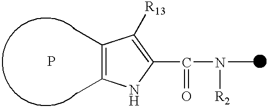
wherein ●, P, R₂, and R₁₃ is a leaving group
c) reacting the product of step (d) with a halide of the formula
R₁ halo
wherein R₁ is as above and halo is a halide 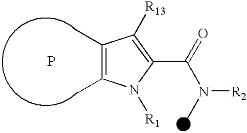
wherein ●, P, R₁, R₂, and R₁₃ are as above
to produce a protected indole of the formula
d) reacting said protected indole produced in step (c) with a boronic acid of the formula 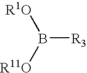
wherein R₃ is as above R¹ and R¹¹ are individually lower alkyl or taken together form a lower alkylene bridge between their attached oxygen atoms
to produce an immobilized compound of the formula I; and 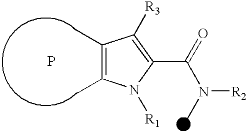
when ●, P, R₁, R₂, and R₃ are as above, and
f) cleaving said immobilized compound of formula I from said solid support to produce the compounds of formula I.

13. A method of preparing a combinatorial library which comprises a plurality of different indoles having the formula: 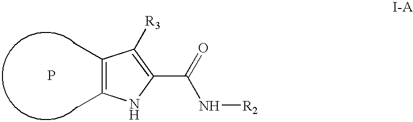
wherein P is a fused ring substituent, which ring substituent is an aromatic ring, a heteroaromatic ring or a cycloaliphatic ring which may be substituted or unsubstituted; R₂ is individually is hydrogen, lower alkyl containing from 1 to 7 carbon atoms, lower alkenyl containing from 2 to 7 carbon atoms, lower alkynyl containing from 3 to 7 carbon atoms, mono and bicycloaliphatic ring with each ring having from 3 to 7 carbon atoms, aryl containing from 1 to 3 fused aromatic rings each ring consisting of 6 carbon atoms, heterocycloaliphatic containing 1 to 2 fused rings with each ring containing from 3 to 6 carbon atoms with one or two hetero atoms selected from the group consisting of O, S and N, monocyclic or bicyclic heteroaryl rings each containing from 3 to 6 carbon atoms with 1 to 4 hetero atoms which can be N, S or O with the proviso that when the hetero atom is S or O, there are 1 or 2 hetro atoms in the ring and when the hetero atoms is N, there are from 1 to 4 N atoms in the ring, and wherein the hetero ring in the heterocycloaliphatic ring or monocyclic or bicyclic heteroaryl rings can be condensed with an aryl, or cycloaliphatic ring and wherein any of the heteroaryl, aryl cycloaliphatic or heteroaliphatic rings in the cyloaliphatic aryl, heteroaryl or heteroaliphatic substituents may be connected to the formula I by a lower alkylene chain containing from 1 to 7 carbon atoms and R₃ is ring substituent selected from the group consisting of an aromatic ring, a heteroaromatic ring and a cycloaliphatic ring which ring substituent may be substituted or unsubstituted,
comprising:
a) immobilizing on a solid support an amine of the formula
R₂NH₂
wherein R₂ is as above
to produce an immobilized amine of the formula: 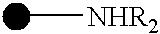
wherein ● is the solid support, and R₂ is as above
b) coupling said immobilized amine to an organic acid of the formula: 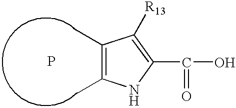
where P is as above, and R₁₃ is a leaving group,
to produce an immobilized amide having a free amino group of the formula: 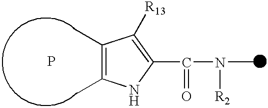
where ●, P, R₂, and R₁₃ is a leaving group
c) reacting the product of step b) with an amino protecting group:
to produce an immobilized amine of the formula 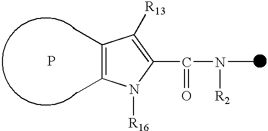
wherein, P, R₂, R₁₃ and ● are as above; and R₁₆ is a hydrolyzable amino protecting group
d) reacting said protected immobilized amide with a boronic acid of the formula: 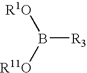
wherein R₃ is as above R¹ and R¹¹ are individually lower alkyl or taken together form a lower alkylene bridge between their attached oxygen atoms
to produce a protected immobilized indole of the formula: 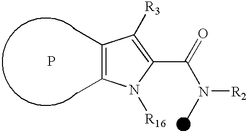
wherein ●, P, R₂, and R₃ is as above and R₁₆ is hydrolyzable amino protecting group
e) and cleaving by hydrolysis the immobilized indole from said solid support to produce the indole of formula I-A.

14. A method of preparing a combinatorial library which contains a plurality of different immobilized indoles having the formula: 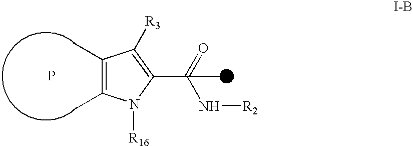
wherein ● is a solid support, P is a fused ring substituent, which ring substituent is an aromatic ring, a heteroaromatic ring or a cycloaliphatic ring which may be substituted or unsubstituted R₂ is hydrogen, lower alkyl containing from 1 to 7 carbon atoms, lower alkenyl containing from 2 to 7 carbon atoms, lower alkynyl containing from 3 to 7 carbon atoms, mono and bicycloaliphatic ring with each ring having from 3 to 7 carbon atoms, aryl containing from 1 to 3 fused aromatic rings each ring consisting of 6 carbon atoms, heterocycloaliphatic containing 1 to 2 fused rings with each ring containing from 3 to 6 carbon atoms with one or two hetero atoms selected from the group consisting of O, S and N, monocyclic or bicyclic heteroaryl rings each containing from 3 to 6 carbon atoms with 1 to 4 hetero atoms which can be N, S or O with the proviso that when the hetero atom is S or O, there are 1 or 2 hetro atoms in the ring and when the hetero atoms is N, there are from 1 to 4 N atoms in the ring, and wherein the hetero ring in the heterocycloaliphatic ring or monocyclic or bicyclic heteroaryl rings can be condensed with an aryl, or cycloaliphatic ring and wherein any of the heteroaryl, aryl cycloaliphatic or heteroaliphatic rings in the cycloaliphatic aryl, heteroaryl or heteroaliphatic substituents may be connected to the formula I by a lower alkylene chain containing from 1 to 7 carbon atoms and R₃ is ring substituent selected from the group consisting of an aromatic ring, a heteroaromatic ring and an cycloaliphatic ring which ring substituent may be substituted or unsubstituted; and R₁₆ is an amino protecting group, comprising:
a) providing on a solid support an immobilized amide of the formula 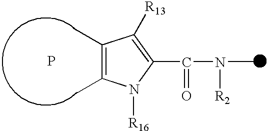
wherein P, R₂, as above and R₁₃ is a leaving group, and R₁₆ is a hydrolyzable amino protecting group
b) reacting said immobilized amide with a boronic acid of the formula 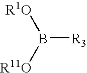
when R₃ is as above R₁ and R₁₁ are individually lower alkyl or taken together form a lower alkylene bridge between their attached oxygen atoms,
to produce said immobilized indole.

15. A method of preparing a combinatorial library comprising a plurality of indole of the formula: 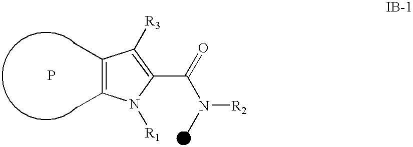
wherein ● is a solid support, P is a fused ring substituent, which ring substituent is an aromatic ring, a heteroaromatic ring or a cycloaliphatic ring which may be substituted or unsubstituted; R₁ and R₂ are individually hydrogen, lower alkyl containing from 1 to 7 carbon atoms, lower alkenyl containing from 2 to 7 carbon atoms, lower alkynyl containing from 3 to 7 carbon atoms, mono and bicycloaliphatic ring with each ring having from 3 to 7 carbon atoms, aryl containing from 1 to 3 fused aromatic rings each ring consisting of 6 carbon atoms, heterocyloaliphatic containing 1 to 2 fused rings with each ring containing from 3 to 6 carbon atoms with one or two hetero atoms selected from the group consisting of O, S and N, monocyclic or bicyclic heteroaryl rings each containing from 3 to 6 carbon atoms with 1 to 4 hetero atoms which can be N, S or O with the proviso that when the hetero atom is S or O, there are 1 or 2 hetro atoms in the ring and when the hetero atoms is N, there are from 1 to 4 N atoms in the ring, and wherein the hetero ring in the heterocycloaliphatic ring or monocyclic or bicyclic heteroaryl rings can be condensed with an aryl, or cycloaliphatic ring and wherein any of the heteroaryl, aryl cycloaliphatic or heteroaliphatic rings in the cycloaliphatic aryl, heteroaryl or heteroaliphatic substituents may be connected to the formula I by a lower alkylene chain containing from 1 to 7 carbon atoms with the further proviso that R₁ is other than hydrogen, and R₃ is ring substituent selected from the group consisting of an aromatic ring, a heteroaromatic ring and an cycloaliphatic ring which ring substituent may be substituted or unsubstituted;
comprising:
a) providing an immobilized amine of the formula: 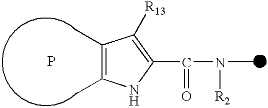
wherein ●, P, and R₂, are as above and R₁₃ is a leaving group
b) reacting the product of step (a) with a halide of the formula
R₁ halo
wherein R₁ is as above and halo is a halide
to produce an immobilized indole product of the formula 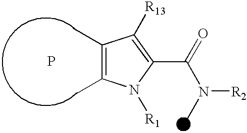
wherein ●, P, R₁, and R₁₃ are as above
c) reacting said protected immobilized indole product of step (b) with a boronic acid of the formula: 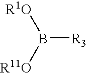
wherein R₃ is as above R₁ and R₁₁ are individually lower alkyl or taken together form a lower alkylene bridge between their attached oxygen atoms
to produce said indole of formula IB-1.

16. A method of producing an immobilized indole of the formula: 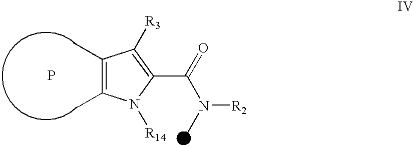
wherein ● a solid support, P is a fused ring substituent, which ring substituent is an aromatic ring, a heteroaromatic ring or a cycloaliphatic ring which may be substituted or unsubstituted; R₂ is a substituent selected from the group consisting of hydrogen, lower alkyl containing from 1 to 7 carbon atoms, lower alkenyl containing from 2 to 7 carbon atoms, lower alkynyl containing from 3 to 7 carbon atoms, mono or bi-cycloaliphatic ring with each ring having from 3 to 7 carbon atoms, aryl containing from 1 to 3 fused aromatic rings each ring consisting of 6 carbon atoms, heterocyloaliphatic containing 1 to 2 fused rings with each ring containing from 3 to 6 carbon atoms with one or two hetero atoms selected from the group consisting of O, S and N, monocyclic or bicyclic heteroaryl rings each containing from 3 to 6 carbon atoms with 1 to 4 hetero atoms which can be N, S or O with the proviso that when the hetero atom is S or O, there are 1 or 2 hetro atoms in the ring and when the hetero atoms is N, there are from 1 to 4 N atoms in the ring, and wherein the hetero ring in the heterocycloaliphatic ring or monocyclic or bicyclic heteroaryl rings can be condensed with an aryl, or cycloaliphatic ring and wherein any of the heteroaryl, aryl cycloaliphatic or heteroaliphatic rings in the cyloaliphatic aryl, heteroaryl or heteroaliphatic substituents may be connected to the formula I by a lower alkylene chain containing from 1 to 7 carbon atoms and R₃ is ring substituent selected from the group consisting of an aromatic ring, a heteroaromatic ring and an cycloaliphatic ring which ring substituent may be substituted or unsubstituted, R₁₄ is an amino protecting group or a substituent selected from those substituents set forth for R₂ except hydrogen,
comprising reacting a compound of the formula: 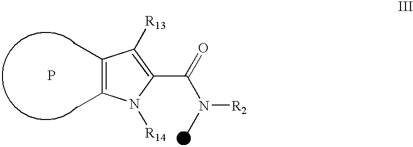
wherein ● is a solid support, R₁₃ is a leaving group; R₁₄ is an amino protecting group, R₁₄, R₂, and P are as above,
with a boronic acid of the formula: 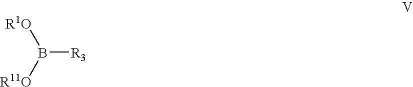
wherein R₃ is as above R¹ and R¹¹ are individually lower alkyl or taken together form a lower alkylene bridge between their attached oxygen atoms, to produce said immobilized amide.

17. The process of claim 16 wherein said leaving group is halide.

18. The process of claim 17 wherein said leaving group is iodo.

19. The process of claim 18 wherein said reaction is carried by a Suzuki reaction.
